(19) United States
(12) Patent Application Publication
(10) Pub. No.: US 2021/0025881 A1
(43) Pub. Date: Jan. 28, 2021

(54) CONVECTIVE FLOW-DRIVEN MICROFABRICATED HYDROGELS FOR RAPID BIOSENSING

(71) Applicant: UNIVERSITY OF WYOMING, Laramie, WY (US)

(72) Inventors: John OAKEY, Laramie, WY (US); Mark H. HARPSTER, Laramie, WY (US); Cheng CHENG, Laramie, WY (US)

(21) Appl. No.: 16/936,208

(22) Filed: Jul. 22, 2020

Related U.S. Application Data
(60) Provisional application No. 62/876,827, filed on Jul. 22, 2019.

(51) Int. Cl. G01N 33/543; G01N 33/58; G01N 21/65

(52) U.S. Cl. CPC G01N 33/54366; G01N 2333/165; G01N 21/658; G01N 33/582

(57) ABSTRACT

Embodiments of the present disclosure generally relate to apparatus and methods for analyte detection. More specifically, embodiments of the present disclosure relate to a microscale biosensing platform based upon the rehydration-mediated swelling of functionalized hydrogel structures and rapid capture of target analyte(s) by induced convective flow. In an embodiment is provided an apparatus for analyte detection that includes a fluidic channel coupled to a substrate and a hydrogel structure coupled to the fluidic channel. The hydrogel structure includes one or more surface-functionalized hydrogel features, wherein at least one of the one or more surface-functionalized hydrogel features comprises a chemically-bound probe, the chemically-bound probe to bind an analyte, and wherein the hydrogel structure is at least partially dehydrated. Apparatus described herein can also include a detector. Methods of detecting analytes are also described herein.

### CROSS-REFERENCE TO RELATED APPLICATIONS

This application claims priority to U.S. Provisional Patent Application Ser. No. 62/876,827 filed on Jul. 22, 2020, which is incorporated herein by reference in its entirety.

### BACKGROUND

### Field

Embodiments of the present disclosure generally relate to apparatus and methods for analyte detection. More specifically, embodiments of the present disclosure relate to a microscale biosensing platform based upon the rehydration-mediated swelling of functionalized hydrogel structures and rapid capture of target analyte(s) by induced convective flow.

### Description of the Related Art

Hydrogel matrices are typically micron-scale macromolecular networks derived from the polymerization and cross-linking of reactive macromers, e.g., poly(acrylic acid), poly(vinylpyrrolidone), and poly(ethylene glycol), that swell and demonstrate high solubility in aqueous solutions. Their biocompatibility, ease in micro-patterning synthesis, and advancements in the chemical derivatization of reactive macromers for tailored polymer chemistries, have fostered significant progress in demonstrating the flexibility of hydrogels for precisely tuning desired physical, mechanical, and chemical properties. The functionalization of hydrogels with biomolecules has facilitated their utilization as biomimetic scaffolds for cell and tissue growth, encapsulants for controlled drug delivery and versatile biosensors for protein, antibody, nucleic acid, and microbe detection. Due to their structural flexibility, hydrogels can undergo osmotic-driven conformational changes in structure (e.g., swelling and shrinkage), the extent of which is dictated by, at least, the respective degree of cross-linkage and charge density of the hydrogel polymer matrix. A broad spectrum of hydrogel sensing platforms have been developed by utilizing the acute responsiveness to changes in the local aqueous environment.

While phase transitions in the hydration state of hydrogels have been exploited for developing a broad spectrum of actuators, the transition that occurs from the dehydrated state to full hydration, and vice-versa, has attracted limited attention. For example, lyophilized spotted arrays of anti-albumin IgG-functionalized hydrogel scaffolds were primarily developed for long-term storage and subsequent use as an albumin capture platform in sandwich immunoassays. Conversely, the dehydration of hydrogel scaffolds containing immobilized Au and Ag nanoparticles previously incubated with a Raman-active pesticide (e.g., sumithion) has been developed as an assay platform for providing a strong surface-enhanced Raman effect. Here, drying and collapse of the hydrated hydrogel matrix served to concentrate captured pesticide within the collective surface metal plasmon resonances induced by laser excitation, thereby greatly increasing the intensity of the Raman signaling and detection sensitivity. In a functionally similar approach, the incubation of tumor necrosis factor alpha (TNF-α) antigen with hydrated acrylamide scaffolds functionalized with anti-TNF-α antibodies and spotted on silicon wafers, after which the capture complex was dried and analyzed by array imaging reflectrometry. Detection sensitivities as low as 1 pg/mL were recorded based on measured increases in wafer thickness. While high-level detection sensitivity was achieved in both cases, instrumentation requirements and the lengthy times required to dry assay reactions are impractical for on-site or point-of-care test applications.

Conventional analyte detection methods typically have slow rates of diffusive mass transfer and capture surface and analyte interaction in solutions of biomolecules with small diffusion coefficients. Recorded assay times range from several minutes to several hours and oftentimes require complex procedures and instrumentation for conducting assays and data processing. Moreover, the ability to rapidly detect low amounts of analytes (e.g., picomolar concentrations) using point-of-care tests remains elusive.

Therefore, there is a need in the art for improved apparatus and methods for rapid analyte detection.

### SUMMARY

Embodiments of the present disclosure generally relate to apparatus and methods for analyte detection. More specifically, embodiments of the present disclosure relate to a microscale biosensing platform based upon the rehydration-mediated swelling of functionalized hydrogel structures and rapid capture of target analyte(s) by induced convective flow.

In an embodiment is provided an apparatus for analyte detection. The apparatus includes a fluidic channel coupled to a substrate. The apparatus further includes a hydrogel structure coupled to the fluidic channel. The hydrogel structure includes one or more surface-functionalized hydrogel features, wherein at least one of the one or more surface-functionalized hydrogel features comprises a chemically-bound probe, the chemically-bound probe to bind an analyte, and wherein the hydrogel structure is at least partially dehydrated.

In another embodiment is provided an apparatus for analyte detection. The apparatus includes a first component and a second component. The first component includes a fluidic channel coupled to a substrate. The first component further includes a hydrogel structure coupled to the fluidic channel, the hydrogel structure comprising one or more surface-functionalized hydrogel features, wherein at least one of the one or more surface-functionalized hydrogel features comprises a chemically-bound probe, the chemically-bound probe to bind an analyte, and wherein the hydrogel structure is at least partially dehydrated. The second component includes a detector to detect the presence or absence of a material detectable by Raman spectroscopy.

In another embodiment is provided a method of detecting an analyte. The method includes introducing a sample to an apparatus described herein, the sample comprising an analyte. The method further includes exposing one or more surface-functionalized hydrogel features of the apparatus to the sample, and concentrating the analyte adjacent to or on the one or more surface-functionalized hydrogel features.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a perspective view of an example apparatus for analyte detection according to at least one embodiment of the present disclosure.

FIG. 1B is a top view of an example apparatus for analyte detection according to at least one embodiment.

FIG. 1C is an illustration of an example surface-functionalized hydrogel feature according to at least one embodiment of the present disclosure.

FIG. 1D is an illustration of the underlying principle of an example assay described herein according to at least one embodiment of the present disclosure.

FIG. 2 is an example apparatus for detecting SARS- and SARS-related coronaviruses according to at least one embodiment of the present disclosure.

FIG. 3 is a flowchart of a method for detecting an analyte according to at least one embodiment of the present disclosure.

FIG. 4 is a rendering of an example fabrication strategy for apparatus described herein fabricated according to at least one embodiment.

FIG. 5A shows fluorescent images of the results acquired for an example convective flow assay according to at least one embodiment of the present disclosure.

FIG. 5B shows fluorescent images of the absence on non-specific protein binding for an example convective flow assay according to at least one embodiment of the present disclosure.

FIG. 5C is a schematic illustrating an example of fluorescence capture at surface-functionalized hydrogel surfaces according to at least one embodiment of the present disclosure.

FIG. 5D is a graph illustrating the kinetics of an example convective flow-mediated capture rate to the rate obtained for mass diffusion according to at least one embodiment of the present disclosure.

FIG. 6 illustrates mass inclusion and exclusion of rhodamine-conjugated proteins using example surface-functionalized hydrogel features according to at least one embodiment of the present disclosure.

FIG. 7A is a graph illustrating example effects that changes in the matrix density of biotin have on the relative level of Neutravidin-rhodamine capture and detection according to at least one embodiment of the present disclosure.

FIG. 7B is a graph illustrating example fluorescence intensity measurements with respect to a range of Neutravidin-rhodamine concentrations according to at least one embodiment of the present disclosure.

FIG. 8A is a graph illustrating example fluorescence intensity measurements for hydrogel features synthesized with varying diameters according to at least one embodiment of the present disclosure.

FIG. 8B shows detailed images of example 200-μm hydrogel features using point-scanning confocal microscopy according to at least one embodiment of the present disclosure.

FIG. 9A is a fluorescent image of example antibody-grafted features according to at least one embodiment of the present disclosure.

FIG. 9B is a fluorescent image showing the absence of non-specific surface interactions according to at least one embodiment of the present disclosure.

FIG. 9C is a schematic illustrating fluorescence capture at outer post surfaces via IgG-functionalized hydrogel posts and pA/G-rhodamine according to at least one embodiment of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to apparatus and methods for analyte detection. More specifically, embodiments of the present disclosure relate to a microscale biosensing platform based upon the rehydration-mediated swelling of functionalized hydrogel structures and rapid capture of target analyte(s) by induced convective flow. The inventors have discovered new and improved methods and apparatus for detecting components of interest (e.g., analytes) that overcome deficiencies of conventional detection tools and methods. The apparatus and methods provided herein address major deficiencies of current diagnostic tools and methods by being able to, at least, analyze small amounts of analytes (e.g., antigens) and reduce analysis time. The apparatus and methods described herein enable in-situ, real-time, and/or rapid diagnosis. Moreover, the apparatus described herein is a single device and/or a disposable device, such as a microfluidic device. The apparatus described herein is also a portable, hand-held device which enhances adoption and utilization in the field or at the point of care.

Reduction in the analysis time arises from, at least, the ability to quickly concentrate analyte(s) in solution and the rehydration of the hydrogel structures. The rehydration induces convective flow and overcomes the long wait times associated with diffusion limited binding (that is, waiting for two molecules or a molecule and a particle to find each other by diffusing through solution). Other techniques can concentrate analytes. For example, SERS nanoparticle assays can be made with magnetic particles that can be pelleted, but the incubation is not accelerated by this step. One must still wait for diffusion-limited binding to occur. Then, the concentration only produces a signal enhancement. Another example is concentration by bulk convective flow in lateral flow immunoassay strips. Here, concentration of the analyte is achieved by washing a large quantity of sample over a capture surface. But, this technique is slow and limited in its ability to concentrate. The methods and apparatus described herein is a local, convection-driven concentration that is both very fast (by nature of the small feature sizes involved) and concentrates to produce a signal enhancement.

Briefly, the apparatus includes a hydrogel matrix having surface-functionalized hydrogel features that serve to, at least, concentrate and retain specific analyte(s). The hydrogel matrix and surface-functionalized hydrogel features also induce convective flow to their surfaces. The surface-functionalized hydrogel features are rapidly fabricated by photolithography using photolabile monomers to form hydrogel features. Subsequently, the hydrogel features are functionalized with capture molecules (e.g., probes) by a variety of chemical methods, such as acrylate chemistry, and then dehydrated for storage. The probes bind, or bond to, analyte(s) of interest in the sample. Upon rehydration with a sample solution containing the analyte of interest, convection-driven flow of the sample solution into the re-hydrating hydrogel matrix draws the analyte(s) towards the functionalized capture surface, concentrating it quickly and passively. For example, following introduction of the sample solution to the apparatus, the analyte(s) flow through the fluidic channel of the apparatus and become concentrated at or near the surface-functionalized hydrogel features. The analyte(s) can then be detected by, e.g., fluorescence, Raman spectroscopy, and UV-Vis spectroscopy.

The apparatus and methods described herein are not limited by sample preparation techniques, e.g., cell lysis and extraction of analytes of interest. Moreover, the apparatus and methods described herein are not limited by the method of detection. Although some of the disclosure herein is provided in the context of antigen detection, it is to be understood that embodiments herein can be used to detect any type of analyte to which a chemical tag (e.g., a fluorescent marker or other chemo- or litho-detectable marker) is attached. For example, apparatus and methods herein can be used to detect analytes such as biologic molecule types, such as proteins, antibodies, nucleic acid sequences (e.g., mRNA), etc. Accordingly, the presentation of portions of the disclosure in the context of antigens is not intended to limit the scope of the present disclosure. Moreover, embodiments herein are used to detect any type of analyte to which an element or molecule itself has a detectable signature, e.g., a Raman spectrum, such as Au nanoparticles and/or Ag nanoparticles. Such detectable analytes include, but are not limited to, biologic molecule types, such as proteins, antigens, antibodies, nucleic acids, etc. Other molecules that have a detectable Raman shift include Raman dyes.

One area of interest for the apparatus and methods described herein include point-of-care diagnostic tests to accurately detect SARS- and SARS-related coronaviruses, such as SARS-CoV-2. Point-of-care diagnostic testing enables testing to be available for communities that cannot readily access laboratory testing and for populations that need to quickly address emerging outbreaks. Major challenges in implementing such point-of-care tests include the speed of the test, their ease of use, the simplicity and economics of their fabrication, and the efficacy of the tests. That is, the point-of-care tests should be rapid and simple such that the tests can serve tens of millions of individuals, should be economically scalable with respect to production of the point-of-care tests, and should be sufficiently accurate and sensitive to reliably detect low levels of viral material present in infected patients.

Conventional diagnostic tests and methods to detect SARS- and SARS-related coronaviruses, however, do not appropriately address these challenges. As an example, certain SARS- and SARS-related coronavirus tests rely on polymerase chain reaction (PCR). However, PCR-based testing requires samples to be mailed to a lab for processing on specialized and expensive equipment. Testing results ultimately take days or weeks to return to the patient. Rapid immunoassay tests are a point-of-care test to detect SARS- and SARS-related coronaviruses that rely on fluorometric or colorimetric detection to visually indicate a patient's infection status. However, existing immunoassay tests are not sufficiently sensitive to meet the needs of a reliable diagnostic for low viral concentrations, and therefore cannot provide a definitive diagnosis.

### Apparatus for Analyte Detection

The present disclosure relates to apparatus (e.g., assays) for analyte detection. The assays are useful for, e.g., the rapid detection of antigens, cells, bacteria, proteins, nucleotides, and viruses such as SARS- and SARS-related coronaviruses. Other molecules can also be detected such as non-biologic molecules, including, but not limited to, explosives. The assays are in the form of a microfluidic device, such as a “lab-on-a-chip” type device. The assay is based upon a rehydration-mediated swelling of surface-functionalized hydrogel features and rapid capture of a target analyte on such features by convective flow, concentrating the target analyte quickly and passively. The apparatus can be free of actuation, such that fluid imbibes into the device spontaneously and concentration of the target analyte can occur without user control. Briefly, to perform the assay, a sample solution including an analyte is introduced to the at least partially dehydrated hydrogel of the assay. Upon rehydration by the sample solution, convection-driven flow of the sample solution into the rehydrating hydrogel matrix draws analyte towards the functionalized surfaces.

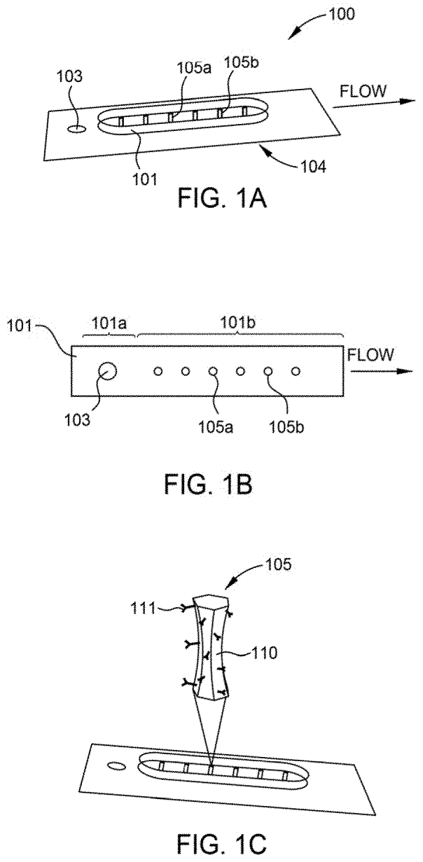

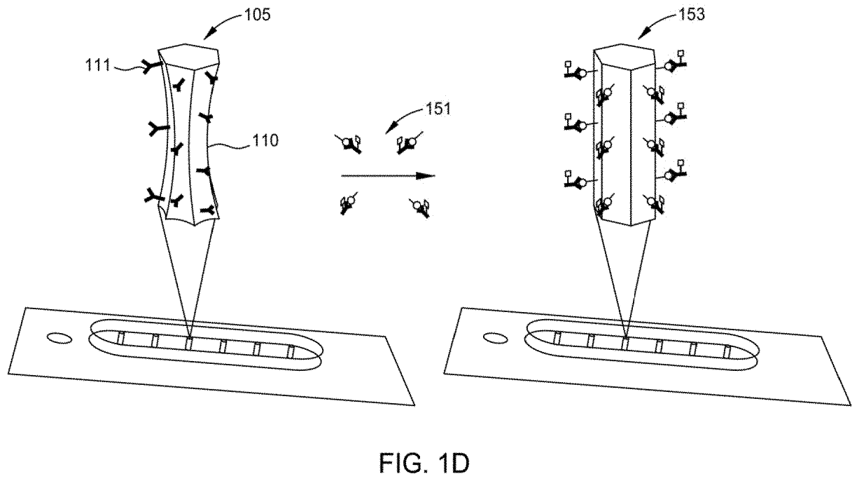

FIG. 1A is a perspective view of an example apparatus 100 for analyte detection according to at least one embodiment of the present disclosure. The apparatus 100 includes a fluidic channel 101. In at least one embodiment, the fluidic channel 101 has a diameter of micrometers (μm) to millimeters, such as at least about 10 μm, such as from about 10 μm to about 2 mm, such as from about 50 μm to about 1 mm or about 10 μm to about 1 mm. FIG. 1B is a top view of the fluidic channel 101 according to at least one embodiment. As shown, the fluidic channel 101 is segregated into at least two components—a sample introduction area 101a and a detection area 101b. The sample introduction area 101a includes a port 103 (e.g., an accessible opening to the fluidic channel 101, such as a sample introduction port) where the sample containing the analyte of interest is introduced. In some embodiments, the port 103 ranges from a few micrometers in diameter to a few millimeters in diameter depending on the application.

The fluidic channel 101 includes a hydrogel structure (or hydrogel matrix). The hydrogel structure is a partially dehydrated (or fully dehydrated) hydrogel matrix that has surface-functionalized hydrogel features. One or more surface-functionalized hydrogel features 105 are coupled to the hydrogel matrix of the fluidic channel 101. The one or more surface-functionalized hydrogel features are also partially dehydrated (or fully dehydrated). The one or more surface-functionalized hydrogel features 105 serve to concentrate and retain the target analyte as the features rehydrate and serve to induce secondary convective flows to their surfaces. Accordingly, the analyte(s) in the sample move from the sample introduction area 101a on the proximal end of apparatus 100 to the detection area 101b on the distal end (in the direction of the arrow). As described below, the surface-functionalized hydrogel features 105 are functionalized with molecule(s) that capture, e.g., bond to, and concentrate analyte(s) of interest for rapid detection. Bonding between an analyte and a surface-functionalized hydrogel feature is accomplished by, e.g., chemical bonding and/or physical bonding.

As stated above, the hydrogel structure is at least partially dehydrated, such as at least about 1% dehydrated, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% dehydrated. In at least one embodiment, the amount of dehydration that the hydrogel structure is dehydrated ranges from d₁ to d₂ (in units of %), where d₁ and d₂ can be, independently, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or about 100, as long as d₁<d₂. In at least one embodiment, the hydrogel structure is at least about 25% dehydrated or at least about 75% dehydrated.

Referring back to FIG. 1A, the port 103 is coupled to the fluidic channel 101. The port 103 can be coupled to the fluidic channel 101 by, for example, chemically bonding, gluing, press sealing, luer-locking, etc. The fluidic channel 101 is affixed to a surface of the substrate 104 (e.g., a glass surface). It is contemplated that a material other than glass can be used as the substrate 104, such as plastics, elastomers, thermoplastics, polyethylene films, polyetheretherketone (PEEK) films, among others. Dimensions of the substrate can be from about 1 mm×1 mm (about 1 mm²) to about 86 mm×54 mm (about 4,700 mm²), such as from about 10 mm² to about 4,500 mm², such as from about 50 mm² to about 4,000 mm².

The one or more surface-functionalized hydrogel features 105 each, independently, include one or more “probe(s)” 111 that physically and/or chemically bond with an analyte as shown in FIG. 1C. The one or more surface-functionalized hydrogel features 105 are located in detection area 101b. Each of the probes 111, independently, is chemically and/or physically attached (bonded) to a part of the hydrogel features 110, e.g., at a location on or near the hydrogel features 110, in order to form the one or more surface-functionalized hydrogel features 105. Each of the probes 111 are the same as or different from the other probes disposed along the fluidic channel 101.

The one or more probe(s) 111 are an element and/or a molecule, e.g., a protein and/or an antibody, that binds a specific analyte as the sample flows through the apparatus 100. In some embodiments, the one or more surface-functionalized hydrogel features 105 include at least one surface-functionalized hydrogel feature 105a having a probe specific for a particular analyte, e.g., a particular antigen. In some embodiments, at least one of the one or more surface-functionalized hydrogel features, e.g., surface-functionalized hydrogel feature 105b, include a probe that binds non-specific analyte(s) to ensure that the test was run. Example methods for fabricating the one or more surface-functionalized hydrogel features 105 are described below.

In some embodiments, the apparatus 100 is included as a component of a kit for analyte detection. The kit can further include other components such as a detector, e.g., a fluorescence detector. The kit can further include buffers for sample preparation, reaction components and reagents, such as chemical tags/markers, e.g., fluorophores and/or a material detectable by Raman spectroscopy, and syringe(s) for introducing the sample to the apparatus 100.

The surface-functionalized hydrogel features 105 are fabricated by photolithography methods using a dilute solution of a photolabile monomer, such as polyethylene glycol diacrylate (PEGDA) monomer, after which the surface of the hydrogel features can be functionalized with probes through various chemical methods such as acrylate chemistry. The probe can be acrylated in order to copolymerize the gel and probe into the same network. Additionally, or alternatively, the hydrogel can be formed and the probe attached afterwards. Other approaches to bond the probe to the hydrogel features include click chemistry (e.g., having a probe with a cysteine group and copolymerizing it or clicking onto a bifunctional acryl-peg-ene), as well as using EDC coupling chemistry (e.g., where probes, such as proteins, can be bound to free hydroxyl groups of the hydrogel).

Photolithography methods include projection lithography, e.g., positive projection lithography (PPL) or negative projection lithography (NPL), to form the hydrogel features. Projection lithography (PL) enables feature fabrication within sealed microfluidic devices by passing UV radiation through a shadow mask placed in the conjugate focal plane of an inverted microscope to polymerize and pattern hydrogel structures within microfluidic channels. To form hydrogel features by PPL, and in some embodiments, a projection mask that enables light to pass through the mask in the polymerization areas of interest is used. After light irradiation polymerizes the hydrogel forming solution, raised hydrogel features on glass are formed. To form hydrogels by NPL, and in some embodiments, a hydrogel forming solution is polymerized to a photodegradable polymer. The polymer is then degraded by exposure to UV light passing through a shadow mask to leave behind raised hydrogel features on the glass surface. After the hydrogel features are formed on the glass slides by PPL and/or NPL, the hydrogel features can be functionalized with chemical structures, e.g., probes, that can bind the analyte of interest, such as antibodies, proteins, peptides, nucleic acids, aptamers, engineered proteins, among others, to form the one or more surface-functionalized hydrogel features 105. The hydrogel is then dehydrated for a period of time to form a partially dehydrated (or fully dehydrated) hydrogel matrix coupled to one or more surface-functionalized hydrogel features 105.

FIG. 1D is an illustration of the underlying principle of an example assay described herein according to at least one embodiment of the present disclosure. This illustration shows a sandwich-type immunoassay depicting rehydration-mediated convective flow delivery of target antigen/reporter complex to an antibody-functionalized hydrogel feature. Here, and as a non-limiting illustration, the hydrogel features 110 are surface-functionalized with antibodies 111 (e.g., immunoglobulin G (IgG)) to form surface-functionalized hydrogel features 105 (e.g., antibody-functionalized hydrogel features). The sample containing the analyte of interest is introduced. As an example, the analyte of interest is an antigen bound to a fluorophore-conjugated antibody 151, such as an analyte (e.g., an antigen) bound to recombinant protein A/G (pA/G)-rhodamine (pA/G-rhodamine). The analyte bound to the fluorophore-conjugated antibody 151 is transported to the surface of the surface-functionalized hydrogel feature 105 for capture by rehydration-mediated convective flow forces in a sandwich immunoassay format resulting in product 153. Of note, the shape of the hydrogel feature can change upon rehydration as shown in FIG. 1D. However, it is also contemplated that the shape of the hydrogel feature can remain unchanged upon rehydration. It is contemplated that other functional groups can be attached to the surface of the hydrogel features, other antigens, other analytes, and other flourophores (or different tagging molecules, such as materials detectable by Raman spectroscopy or SERS) can be used as the above description is a non-limiting illustration.

The surface-functionalized hydrogel features 105 are not limited in terms of morphology. Non-limiting examples of the shape of the surface-functionalized hydrogel features 105 include columns, posts, pillars, cubes, cuboids, prisms, cylinders, cones, spheres, doughnuts, or a combination thereof.

### Example Apparatus for Detecting SARS- and SARS-Related Coronaviruses

Embodiments of the present disclosure also relate to apparatus for detecting SARS- and SARS-related coronaviruses. The apparatus for detecting SARS- and SARS-related coronaviruses provided herein overcomes deficiencies of the current diagnostic tests—e.g., those diagnostic tests based on PCR and rapid immunoassay. Briefly, the apparatus combines a convective flow analyte concentration assay with Raman spectroscopy as the detection modality, specifically surface-enhanced Raman spectroscopy (SERS). These coupled systems—incorporating both an assay and a detector—can significantly enhance detection time relative to conventional apparatus and methods by enabling simultaneous sample incubation and analyte concentration, yielding rapid, sensitive, and specific detection of the SARS-CoV-2 nucleoprotein at low analyte concentrations. Moreover, the portability of the coupled systems enables point-of-care testing.

As compared to conventional colorimetric tests for detecting SARS- and SARS-related coronaviruses, the apparatus described herein detects analyte concentrations of about 3-4 orders of magnitude below the limits of conventional colorimetric tests, or even about 6-8 orders of magnitude below the limits of conventional colorimetric tests. Traditionally, SERS-based assays have been slow due to the utilization of separate sample incubation and reporter concentration steps, thus requiring laboratory-bound instrumentation. As such, SERS has largely remained impractical for conducting point-of-care tests at scale. To overcome these issues, the apparatus includes, e.g., a microfluidic device such as microscale test strips, that automatically induce convective flow to incubate target analytes with binding molecules, while simultaneously concentrating these bound complexes for rapid detection.

The apparatus overcomes the diffusion limitations of traditional SERS assays and the limited sample concentration of lateral flow immunoassays (LFIA). The apparatus further includes a detector, such as a simple, inexpensive, hand-held SERS detector. Together, this point-of-care apparatus enables SERS-enhanced detection of, e.g., SARS-CoV-2 nucleoprotein from, e.g., nasopharyngeal samples, and accurately diagnose individuals with COVID-19, even at low viral titers. In some examples, the point-of-care apparatus is capable of achieving analyte detection in under about 3 minutes with a 10 picomolar (pM) limit of detection. Although embodiments described here are related to SARS- and SARS-related coronaviruses, other viruses can be detected, such as influenza, adenovirus, among others.

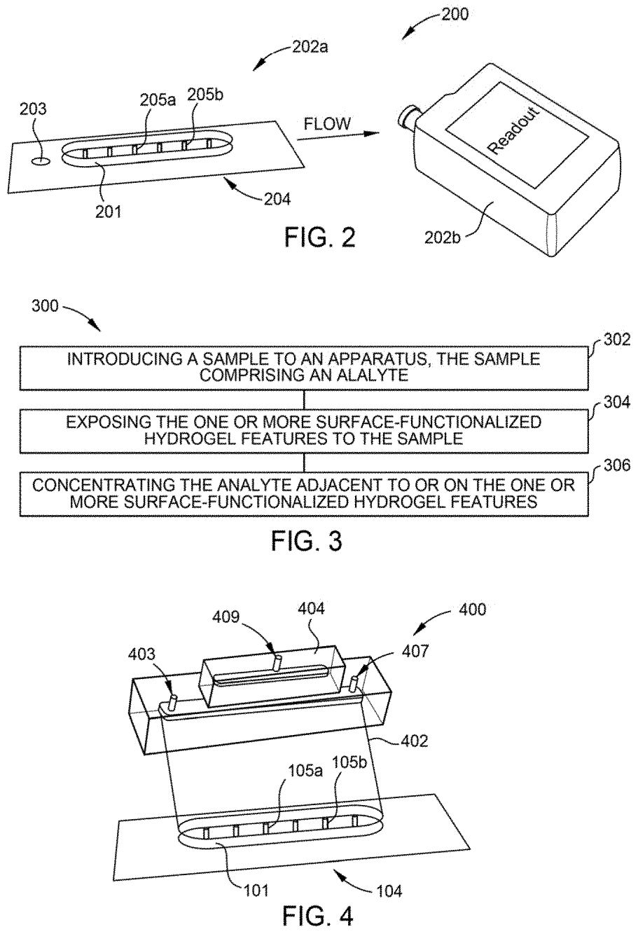

FIG. 2 is an example apparatus 200 for detecting SARS- and SARS-related coronaviruses according to at least one embodiment of the present disclosure. Apparatus 200 includes a first component 202a and a second component 202b. The first component 202a is in the form of a microfluidic device, such as a microfluidic test strip.

The first component 202a includes a fluidic channel 201. In at least one embodiment, the fluidic channel 201 has a diameter of micrometers to millimeters, such as at least about 10 μm, such as from about 10 μm to about 2 mm, such as from about 50 μm to about 1 mm or about 10 μm to about 1 mm. The fluidic channel 201 is segregated into at least two areas—a sample introduction area and a detection area (not shown), similar to that as shown in FIG. 1B. The sample introduction area includes a port 203 (e.g., an accessible opening to the fluidic channel 201, such as a sample introduction port) where the sample containing the analyte of interest is introduced. In some embodiments, the port 203 ranges from a few micrometers in diameter to a few millimeters in diameter depending on the application. The port 203 is coupled to fluidic channel 201. The port 203 can be coupled to the fluidic channel 201 by, for example, chemically bonding, gluing, press sealing, luer-locking, etc. The fluidic channel 201 is affixed to a surface of the substrate 204 (e.g., a glass surface). It is contemplated that a material other than glass can be used as the substrate 204, such as plastics, elastomers, thermoplastics, polyethylene films, polyetheretherketone (PEEK) films, among others. Dimensions of the substrate can be from about 1 mm×1 mm (about 1 mm²) to about 86 mm×54 mm (about 4,700 mm²), such as from about 10 mm² to about 4,500 mm², such as from about 50 mm² to about 4,000 mm².

The fluidic channel 201 includes a hydrogel structure (or hydrogel matrix). The hydrogel structure can be a partially dehydrated (or fully dehydrated) hydrogel matrix that has surface-functionalized hydrogel features. One or more surface-functionalized hydrogel features 205 are coupled to the hydrogel matrix of the fluidic channel 201. The one or more surface-functionalized hydrogel features are also partially dehydrated (or fully dehydrated). The one or more surface-functionalized hydrogel features 205 serve to concentrate and retain the target analyte as the features rehydrate and serve to induce secondary convective flows to their surfaces. Accordingly, the analyte(s) in the sample move from the sample introduction area on the proximal end of the first component 202a to the detection area on the distal end of the first component 202a (in the direction of the arrow). The surface-functionalized hydrogel features 205 are functionalized with molecule(s) that capture, e.g., bond to, and concentrate analyte(s) of interest for rapid detection. Bonding between an analyte and a surface-functionalized hydrogel feature is accomplished by, e.g., chemical bonding and/or physical bonding.

As stated above, the hydrogel structure is at least partially dehydrated, such as at least about 1% dehydrated, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% dehydrated. In at least one embodiment, the amount of dehydration that the hydrogel structure is dehydrated ranges from d₃ to d₄ (in units of %), where d₃ and d₄ can be, independently, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or about 100, as long as d₃<d₄. In at least one embodiment, the hydrogel structure is at least about 25% dehydrated or at least about 75% dehydrated.

The one or more surface-functionalized hydrogel features 205 each, independently, include one or more “probe(s)” that physically and/or chemically bond with an analyte, similar to that as shown for apparatus 100 in FIG. 1C. The one or more surface-functionalized hydrogel features 205 are located in detection area 201b. Each of the probes is, independently, chemically and/or physically attached (bonded) to a part of the hydrogel features, e.g., at a location on or near the hydrogel features, in order to form the one or more surface-functionalized hydrogel features 205. Each of the probes are the same as or different from the other probes. The one or more probe(s) are an element and/or a molecule, e.g., a protein and/or an antibody, that binds a specific analyte as the sample flows through the first component 202a of apparatus 200. In some embodiments, the one or more surface-functionalized hydrogel features 205 include at least one surface-functionalized hydrogel feature 205a having a probe specific for a particular analyte, e.g., a particular antigen. In some embodiments, at least one of the one or more surface-functionalized hydrogel features, e.g., surface-functionalized hydrogel feature 205b, can include a probe that binds non-specific analyte(s) to ensure that the test was run.

Apparatus 200 further includes a second component 202b. The second component 202b is or includes a detector, such as a SERS detector, e.g., a compact, hand-held SERS reader. The first component 202a integrates with the second component 202b for point-of-care detection.

As described above, the surface-functionalized hydrogel features 205 is fabricated via photolithography methods using a dilute solution of a photolabile monomer, such as polyethylene glycol diacrylate (PEGDA) monomer. After hydrogel features are grown, the surface of the hydrogel features are then functionalized with a probe that selectively binds to the analyte of interest. For detection of SARS-CoV-2, the surface-functionalized hydrogel feature 205a includes a probe, such as an antibody or engineered binding molecules that specifically bind to spike (S) proteins of SARS-CoV-2, nucleoproteins (N) of SARS-CoV-2, or a combination thereof.

Prior to introduction of the sample to the apparatus for detecting an analyte, the sample is prepared for analyte detection. For example, the sample is mixed with a buffer, e.g., a phosphate-buffered saline (PBS) buffer. As an example, a nasopharyngeal swab from a patient is mixed with a buffer to form the sample. Additionally or alternatively, and prior to introduction of the sample to the first component 202a of apparatus 200, the analyte(s) of the sample is tagged with a tagging material, e.g., an element or molecule such as a material detectable by Raman spectroscopy (e.g., a gold nanoparticle), to form a tagged analyte. The chemical tag enables detection of the analyte of interest. As an example, the sample is reacted, under reaction conditions, with functionalized gold nanoparticles, that bond to the analyte indicative of SARS-CoV-2 (e.g., spike proteins of SARS-CoV-2, nucleoproteins of SARS-CoV-2, or a combination thereof) to form the tagged SARS-CoV-2 analyte. The gold nanoparticles enable detection of the SARS-CoV-2 analyte by SERS. Accordingly, and in some embodiments, example apparatus 200 further include buffers for sample preparation, reaction components and reagents, such as materials detectable by Raman spectroscopy or SERS, e.g., functionalized gold nanoparticles, as well as syringes for introducing the sample to the first component 202a.

Briefly, as the tagged SARS-CoV-2 analyte flows through the fluidic channel 201 of the first component 202a, the tagged SARS-CoV-2 analyte is captured by surface-functionalized hydrogel features 205a specific for SARS-CoV-2 and the bound analyte is concentrated at the surface. Other non-specific analytes continue to flow through the fluidic channel of the first component 202a. These latter analytes can be captured by, e.g., surface-functionalized hydrogel features 205b functionalized with, e.g., control antibodies, to ensure that the test was run. After a pre-determined period of time, the first component is placed within the second component which scans the first component and returns, for example, a yes/no (+/−) result indicating the presence or absence of the analyte of interest.

In some examples, the apparatus 200 is capable of achieving detection in under 3 minutes with a 10 pM limit of detection.

### Methods for Detecting an Analyte

Embodiments of the present disclosure also relate to methods for analyte detection. For example, the convective flow assay, e.g., apparatus 100 or the first component 201a of apparatus 200, is used to determine the presence or absence of an analyte of interest within a sample, e.g., proteins, antigens, viruses, bacteria, antibodies, nucleic acids, peptides, etc., and non-biologic molecules. These components can be minor constituents of the sample and can therefore be challenging to detect by conventional methods.

FIG. 3 is a flowchart of a method 300 for detecting an analyte according to at least one embodiment of the present disclosure. The method 300 is used with apparatus 100 or apparatus 200, although apparatus 100 and apparatus 200 are only examples of apparatus that may be used in conjunction with method 300. The method 300 includes introducing a sample (e.g., a solution containing analyte(s) of interest) to the apparatus, e.g., apparatus 100, at operation 302.

Prior to introduction of the sample to the apparatus for detecting an analyte, the sample is prepared for analyte detection. For example, the sample is mixed with a buffer, e.g., a phosphate-buffered saline (PBS) buffer. As an example, a nasopharyngeal swab from a patient is mixed with a buffer to form the sample. Additionally or alternatively, and prior to introduction of the sample to an apparatus for detecting an analyte, the analyte(s) of the sample are tagged with, e.g., an element or molecule such as a fluorescent molecule or a material detectable by Raman spectroscopy (e.g., a gold nanoparticle), to form a tagged analyte. The chemical tag enables detection of the analyte of interest. That is, detectors such as a fluorescent detector or a SERS detector are used to detect the presence or absence of the chemical tag. Forming the tagged analyte includes introducing components and/or reaction mixture precursors that, upon interaction with the sample, react the analyte of interest with a chemical tag under reaction conditions. As an example, functionalized nanoparticles (e.g., gold nanoparticles, silver nanoparticles, etc.) or a fluorescent molecules (e.g., fluorescein, Texas red, mCherry, rhodamine, etc.) are mixed with the analyte of interest. The functionalized gold nanoparticles are detectable by Raman spectroscopy, while the fluorescent molecule are detectable by fluorescence. When the tagged analyte interacts with the probe 111 of the one or more surface-functionalized hydrogel features 105, the tagged analyte becomes concentrated on the one or more surface-functionalized hydrogel features 105 and a determination of the analyte present can be made. As described above, and as an example, the surface-functionalized hydrogel features, e.g., surface-functionalized hydrogel features 105, include at least one surface-functionalized hydrogel feature 105a having a probe specific for a particular analyte, e.g., a particular antigen, and at least one surface-functionalized hydrogel feature 105b having a probe that binds non-specific analyte(s) to ensure that the test was run.

The method 300 further includes exposing one or more surface-functionalized features to the sample at operation 304, and concentrating the analyte(s) at a location adjacent to or on the surface-functionalized hydrogel features at operation 305. Here, convective flow forces the sample through the apparatus to the surface-functionalized hydrogel features that are specifically functionalized to capture the analyte(s) of interest. For example, surface-functionalized hydrogel feature 105a includes a probe specific for a tagged analyte and will not detect other analytes, while surface-functionalized hydrogel feature 105b includes a probe that is not specific for any particular tagged analyte, and thereby act as a control to ensure the test was run. As such, enhanced testing results can be enabled by utilizing the convective flow methodology and surface functionalized hydrogel features by improving testing accuracy, reducing the probability of false-positive tests, and reducing the probability of false-negative tests. Moreover, the test is very rapid. For example, the assays can be performed 10-30× faster than conventional tests with sensitivities comparable to conventional tests.

The method 300 can further include detecting the analyte by, e.g., fluorescence spectroscopy, Raman spectroscopy, or a combination thereof, though the method of detection is not limited to such detection methods. Although the method 300 is described in relation to apparatus 100, it is contemplated that a same or similar method can be used in conjunction with apparatus 200.

The following non-limiting examples show that analyte capture and detection using, e.g., a fluorometric rehydration assay, can be achieved within less than a minute following incubation with analyte-containing solution. The rates of recognition recorded are significantly faster than what is typically reported for conventional immunoassays. Without being bound by theory, the significantly faster rate of recognition is believed to be a direct consequence of rehydration-mediated convective flow, which effectively overcomes the slow rates of diffusive mass transfer and capture surface and analyte interaction in solutions of biomolecules with small diffusion coefficients.

### Examples

The apparatus and methods described herein include the following materials: ethanol, dimethyl sulfoxide (DMSO), 10× phosphate buffered saline (PBS), 3-(trimethyloxysilyl)propyl acetate, ethanolamine, biotin, ovalbumin, β-lactoglobulin A and heterobifunctional Acrylate-PEG-N hydroxysuccinimide (AC-PEG-NHS) (M_{w} 5 kDa), any of which are commercially available from Sigma Aldrich (USA). Neutravidin, NHS-rhodamine and glass slides are available from Thermo Fisher Scientific (USA). Homobifunctional PEG diacrylate (PEGDA 700) (M_{w} 700 Da) and heterobifunctional Acrylate-PEG-Biotin (AC-PEG-Biotin) (M_{w} 2 kDa) are commercially available from Jenkem Technology (USA) and Nanocs (USA), respectively. Polydimethylsiloxane (PDMS) is commercially available from Ellsworth Adhesives (USA) as a kit containing viscous elastomer (part A) and curing cross-linker (part B). Recombinant protein A/G (pA/G) is commercially available from Prospec (Israel) and superoxide dismutase is commercially available from Worthington Chemicals (USA). SU-8 50 epoxy photoresist and silicon wafers are commercially available from MicroChem (USA) and Silicon Inc. (USA), respectively. Rabbit pre-immune serum used to prepare enriched IgG by protein A/G agarose affinity chromatography is commercially available from Thermo Fisher Scientific.

Fluorescent images were acquired using a 100 W Hg lamp and Olympus U-MNG cube with a band pass filter for excitation, and a long pass BA590 barrier filter for red emission detection. Images were recorded using Q-Capture Pro 7™ software (commercially available from Qimaging, USA) control of a Q-Color5™ digital camera imaging system (Olympus). Confocal microscopy was performed using a Zeiss laser scanning confocal microscope (Zeiss LSM 710, 561 nm laser line) equipped with ZEN 2009™ software for operation and X-Cite™ 120Q as the light source. Images were processed using ImageJ software (NIH) for background subtraction and measurement of Absolute Fluorescence Intensities.

### Example 1: Device Fabrication of PDMS Chambers and Hydrogel Micropillar Synthesis

In this example using lithography procedures for patterning, PDMS chambers were fabricated by first mixing a PDMS solution and a curing agent in a 10:1 ratio, which are then applied to a SU8-50 micropatterned silicon wafer and cured overnight in a 70° C. oven. Following removal of the PDMS replicate (channel dimensions: 220 μm (h)×3000 μm (w)×5000 μm (l)), inlet ports and outlet ports were punched using a 20 G needle (the hole made can be slightly smaller than the outside diameter of the needle, e.g., slightly less than about 750 μm or about 3/100″), after which the chamber was sealed by bonding to an acrylated glass slide previously immersed in 3-(trimethoxysilyl)propyl acetate, washed with EtOH, and then dried. Hydrogel features were synthesized by the injection of 30 μL of a prepolymer solution (chamber volume) of 0.02% (w/w) of the photoinitiator lithium phenyl-2,4,6-trimethylbenzolyl-phosphinate (LAP), 20% (w/w) PEGDA 700, and varying concentrations of either AC-PEG-Biotin or AC-PEG-NHS.

The chamber was then placed with the glass slide down on the platform of an Olympus IX81 microscope fitted with a Polygon400 multiwavelength spatial illuminator (commercially available from Mightex, Canada), upon which the selected UV light pattern is projected (10 s, 20× lens/1.6 mW/cm²) and controlled for hydrogel features (e.g., posts) synthesis using integrated PolyScan software. Metamorph software (commercially available from Molecular Devices, USA) was used for imaging and control of platform movement. Following hydrogel posts synthesis, the glass slide was removed and the chamber flushed with PBS, followed by EtOH. Additionally, or alternatively, following post synthesis, the glass slide was removed from the chamber, air-dried overnight, and then reattached to the PDMS chamber for conducting convective flow assays.

FIG. 4 is a rendering 400 of an example fabrication strategy for apparatus described herein fabricated according to at least one embodiment. Specifically, the example is a PDMS/glass chamber. The fluidic channel 101, substrate 104, and surface-functionalized hydrogel features 105 are discussed above. The fluidic channel 101 is coupled to a first chamber 402. The first chamber 402 houses the pre-polymer solution (e.g., 0.02% (w/w) of the photoinitiator lithium phenyl-2,4,6-trimethylbenzolyl-phosphinate (LAP), 20% (w/w) PEGDA 700 and varying concentrations of either AC-PEG-Biotin or AC-PEG-NHS). A second chamber 404 is positioned atop the first chamber 402. The second chamber 404 delivers a steady stream of nitrogen gas, N₂, for the purging of oxygen gas (O₂) during hydrogel features synthesis. Other gases, such as inert or noble gases are utilized in combination with N₂ or in place of N₂ in certain embodiments. Oxygen purging serves to minimize incomplete polymerization upon UV exposure and maximize the structural integrity of microfabricated hydrogel features. It is believed that O₂ is consumed by a photoinitiator in a reaction that generates free radicals and peroxides that inhibit photopolymerization due to the inactivation of acrylate groups. In some embodiments, N₂ purging mitigates this effect by reducing the influx of O₂ diffusion from PDMS into solution during photopolymerization. Inlet port 403 is coupled to first chamber 402 and enables introduction of monomers and PDMS into the first chamber 402. An outlet port 407 is coupled to the first chamber 402. During posts fabrication, the outlet port 407 serves to allow flushing of any unreacted monomer, buffers, etc. out of the chamber.

### Example Protein Labeling of the Hydrogel Features

Neutravidin (NA) and pA/G were conjugated with NHS-rhodamine (Thermo Fisher). The method of conjugation included adding NHS-rhodamine, adjusted to 10 mg/mL in DMSO, to a 10-fold mole excess to 5 mg/mL of protein in 1×PBS. The mole ratio of rhodamine to protein is about 2 to 3. The solution was thoroughly mixed and stored for a minimum of 2 hr at 10° C. Following incubation, unreacted NHS-rhodamine was removed by gravity filtration using a PD-10 desalting column (GE Healthcare, USA), after which the recovered conjugate was concentrated by centrifugation (2000×g) using an Amicon Ultra (3 k, molecular weight cut-off (MWCO)) (MilliporeSigma, USA) filtration unit. Protein determination (Quick-start dye reagent, commercially available from BioRad, USA) and UV-Vis spectroscopy of conjugates were then conducted to determine final protein concentration and labeling efficiency.

### Example 2: Device Fabrication and Surface Functionalization

PDMS (Sylgard™ 184, Dow Corning) devices were replicated from photolithographically-patterned silicon wafers using conventional soft lithography techniques. PEG features were created within PDMS straight channels (5 cm in length and 4 mm in width) with depths of 20 or 50 μm. The thickness of the PDMS devices was approximately 10-20 mm for purged devices and approximately 50-60 mm for ambient devices. Oxygen plasma-treated PDMS channel replicas were bonded to plasma-cleaned glass coverslips. Bonded channels were used for all PPL experiments. For the purged devices, an additional PDMS straight channel was fabricated that was 50 μm in depth and approximately two-thirds the length of the initial channel. This channel was bonded to the top of thin PDMS devices and used to flow nitrogen through to purge the device of oxygen. For NPL experiments, acrylate-modified glass coverslips were used. Briefly, glass coverslips were cleaned using a Bunsen burner and placed in a solution containing 190 proof ethanol (Sigma-Aldrich) and 3-(acryloyloxy)-propyltrimethyloxysilane (APTS, Alfa Aesar). The glass coverslips were removed after 5 minutes, rinsed with 190 proof ethanol, and placed in an oven to dry at 70° C. for a minimum of 20 minutes. PDMS channels were placed in contact with the acrylate-modified coverslips to form a spontaneous, reversible seal that was sufficient to maintain a bond during fluid exchange. Shadow masks used for projection lithography were created in AutoCAD and printed as transparency masks (CAD Art).

### Example 3: Synthesis of Photodegradable PEGDA (PEGdiPDA)

An o-nitrobenzyl acrylate moiety was coupled to PEG-bis-amine (Mn˜3400 Da, Laysan Bio) using the following procedure. Acetovanillone was used to synthesize 4-(4-(1-(acryloyloxy)ethyl)-2-methoxy-5-nitrophenoxy)butanoic acid (o-NB acrylate) through a multistep procedure. The o-NB acrylate (4.4 equiv) was coupled to PEG-bis-amine (1 equiv) using carbodiimide chemistry with carboxylic acid activation using 1-[bis(dimethylamino)methylene]-1H-1,2,3-triazolo[4,5-b]pyridinium3-oxide hexafluorophosphate (HATU, 4.4 equiv) in the presence of N,N-diisopropylethylamine (DIPEA, 8 equiv). The reaction was completed overnight under argon gas at room temperature. The resulting functionalized polymer was precipitated into ethyl ether and obtained through centrifugation. The polymer was purified by dialysis against distilled (DI) water (MWCO 1000 Da) and lyophilized to obtain an orange solid. The photocleavable polymer product, PEGdiPDA, was characterized by ¹H NMR spectroscopy (Bruker Daltonics, 600 Hz, 128 scans, deuterated DMSO) using the protons associated with the acrylate (6.35 ppm) and the amide (7.91 ppm) relative to the PEG backbone (3.5 ppm).

### Example 4: Synthesis of lithium phenyl-2,4,6-trimethylbenzolyl-phosphinate (LAP)

The photoinitiator LAP (Lithium phenyl-2,4,6-trimethylbenzoylphosphinate) was synthesized by the following procedure. 2,4,6-trimethylbenzoyl (1 equiv) was slowly added to dimethylphenylphosphonite (1 equiv) under argon gas at room temperature. The mixture was left to react for 18 h, and then lithium bromide (4 equiv) in 2-butanone (100 mL) was added. The solution was heated to 50° C. until a solid precipitate formed. After precipitate formation, the reaction was cooled to room temperature and subsequently filtered. The filtrate was rinsed 3 times with 2-butanone, and excess solvent was removed under pressure. The dried product, LAP, was characterized using ¹H NMR (Bruker Daltonics, 600 Hz, 128 scans, CDCl₃).

### Example 5: Positive Projection Lithography Procedure

A positive shadow mask was attached to the iris in the field aperture of an inverted microscope (Olympus IX81). The inverted microscope was fitted with a Prior Lumen 200 light source via a liquid light guide. Once the shadow mask was in place, a microfluidic channel was filled with PEGDA hydrogel forming monomer solution and placed on the microscope. The PEGDA hydrogel forming monomer solution was 60% w/w PEGDA (M_{W}=700 Da, Sigma-Aldrich), 1% w/w LAP, 1% v/v 1-vinyl-2-pyrrolidinone (NVP, Sigma-Aldrich), and 1% v/v acryloxyethyl thiocarbamoyl Rhodamine B (Rhodamine, Polysciences). Rhodamine B was used to facilitate focusing for photopolymerization on the microscope. The shadow mask was focused by excitation of Rhodamine B at a wavelength of 550 nm with a Texas Red filter cube (Semrock). The gel was polymerized using a long wavelength UV light (DAPI filter with peak at λ=365 nm, Semrock) with exposure times of 50 milliseconds (ms), 100 ms, 250 ms, and 500 ms. MetaMorph™ Microscopy Automation and Image Analysis Software were used to control the exposure time through an automated shutter (Ludl). Before each experiment was conducted, a test hydrogel was made to adjust for changes in bulb light intensity due to usage. For each test experiment, the microfluidic channel was filled with the hydrogel forming solution, the aperture of the microscope closed, and the sample was exposed to 365 nm light for the exposure times stated above. The microfluidic channel was flushed with PBS, and the hydrogels were imaged and compared to hydrogels formed on previous days. If the hydrogels were not observed or not consistent, the light intensity was adjusted by 1% increments until consistent hydrogel formation was observed. This process was also conducted on several different microscopes to adjust light intensity settings for consistent hydrogel formation.

Three different polymerization conditions were used to form features: (1) an ambient microchannel with features formed under the 20× (numerical aperture=0.45, I₀=407 mW/cm²) objective (ambient 20×); (2) an ambient microchannel with features formed under the 40× (numerical aperture=0.65, I₀=383 mW/cm²) objective (ambient 40×); and (3) a nitrogen-purged microchannel with features formed under the 20× objective (purged). For nitrogen-purged devices, the monomer-filled channel was purged with nitrogen for 15 minutes before starting polymerization and continuously throughout the polymerization.

### Example 6: Negative Projection Lithography Procedure

A microfluidic channel was filled with PEGdiPDA hydrogel forming monomer solution and placed on the microscope. The hydrogel forming monomer solution was 8.2% w/w PEGdiPDA, 1% w/w LAP, and 1% v/v Rhodamine, which was included for aiding in focusing of the shadow mask. A large post structure was polymerized within the microfluidic channel using the 20× objective (numerical aperture=0.45) with the field aperture iris opened to create an exposed region of approximately 700 μm. These regions were created to minimize the amount of hydrogel degradation utilized to pattern isolated hydrogel features. Photopolymerization was achieved by irradiation of focused light passed through a 405 nm long pass filter (LP405, Olympus, I₀=355 mW/cm²) for 300 ms. After post polymerization, the channel was flushed with PBS to remove any unreacted monomer solution. A negative shadow mask was then placed in the field aperture of the inverted microscope and focused as described above. Once the shadow mask was focused, long-wavelength UV light (365 nm, I₀=407 mW/cm²) was used to degrade the post structure leaving behind the desired features. Degradation exposure times of 2500 ms, 5000 ms, and 7500 ms were used.

### Example 7: In Situ Hydrogel Degradation

PEGdiPDA features (e.g., posts or other morphologies described herein) with Rhodamine copolymerized within were fabricated using visible light as described above. In this embodiment, the incorporation of Rhodamine B was used to monitor hydrogel degradation. After post formation, any remaining monomer solution was flushed from the device with buffer, e.g., phosphate buffered saline. Posts were degraded by exposure to long-wavelength UV light for 30 s. The diffusion of Rhodamine into the surrounding fluid was monitored as a measure of the change in the surrounding microenvironment by acquiring a fluorescent image every 30 s for 10 minutes. Image acquisition was controlled by MetaMorph, and the shutter was closed between time points to avoid photo bleaching of the sample. Acquired images were analyzed using a Radial Profile plugin for ImageJ.

### Example 8: Multilayered Features

Photodegradable features (e.g., posts) were formed as described above except nonbonded microfluidic channels were used. The first layer was formed in a channel of 20-μm depth with a PEGdiPDA solution containing Rhodamine. After forming the first layer of posts, the channel was removed, and the posts flushed with PBS. A channel of 50-μm depth was placed over the top of the posts and filled with a PEGdiPDA solution containing AlexaFluor™ 488 (BSA-488, Invitrogen) at 20% v/v in place of Rhodamine. Rhodamine-containing posts were located; the aperture coarsely aligned with the post, and the second layer was polymerized on top of it using the same aperture size and an exposure time of 600 ms. Fine alignment was not required as nonoverlapping edges contributed a fraction of the available structure and could be avoided during final feature formation. The channel was flushed with PBS, and the posts were subsequently degraded by light irradiation passed through a shadow mask to form microstructures using the same procedure described above. Posts were imaged using a Zeiss laser scanning confocal microscope (Zeiss LSM 710) and stitched together using the ImageJ “Volume Viewer” plugin.

### Example 9: Protocol for Analyte Detection

Typically, the tagging molecules (e.g., flourophores or materials detectable by Raman spectroscopy or SERS) are either immobilized in the channel of the apparatus for solvation during sample introduction, or the tagging molecules are mixed with the sample and introduced to the apparatus. After a period of time (e.g., a few minutes), the apparatus can be imaged. An example protocol adopted for conducting convective flow assays includes the injection of phosphate buffered saline (PBS) solution containing rhodamine-conjugated protein analyte into a polydimethylsiloxane (PDMS) chamber affixed to a glass slide upon which an array of functionalized hydrogel posts are fabricated and dried. Subsequent to injection, fluorescent images were acquired for protein analyte recognition over time by monitoring the relative rate of binding at the hydrogel post structures both during and following rehydration.

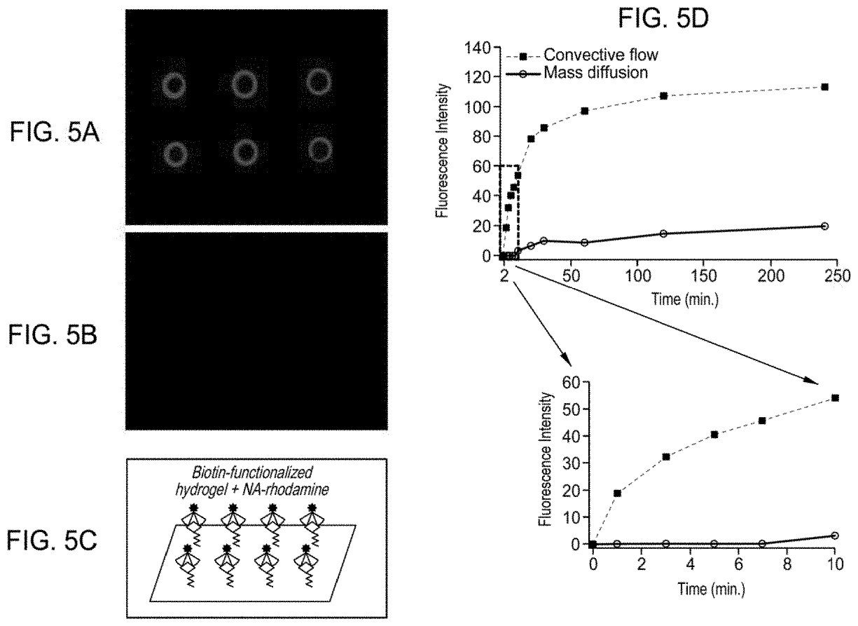

The results acquired for a convective assay utilizing posts fabricated with PEGDA and AC-PEG-Biotin, and NA-rhodamine as the tagged analyte are show in FIG. 5A. FIGS. 5A and 5B show a fluorescent image in the x-y plane (proximal to glass slide) of PEG-DA (20% w/w)/AC-PEG-Biotin hydrogel (0.5% (w/w)) posts rehydrated in PBS containing 0.2 mg/mL NA-rhodamine (top panel, 50 ms exposure acquired 5 minutes subsequent to initiation of reaction) and 0.65 mg/mL pA/G-rhodamine (bottom panel, 500 ms exposure), respectively.

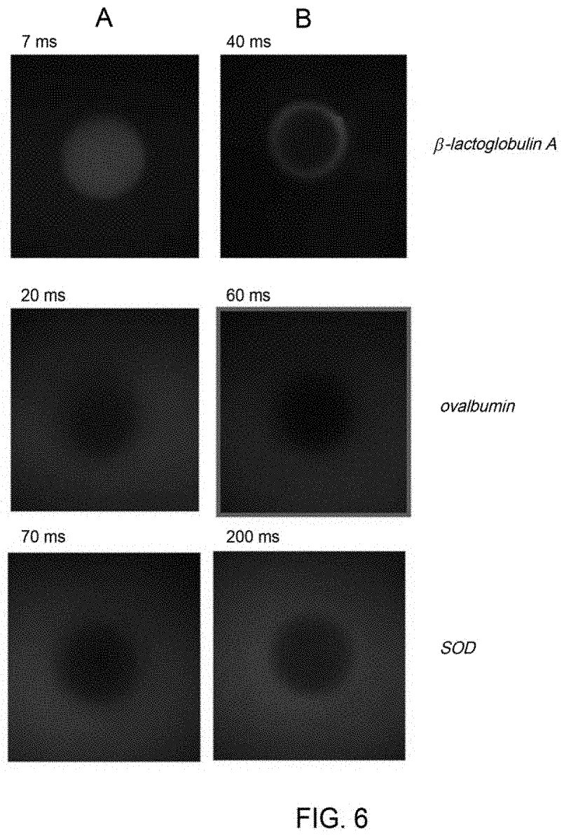

As shown in FIG. 5A, for each post viewed in the x-y plane, intense fluorescence is localized along their circumference following extensive washing with buffer, which is indicative of NA-biotin binding at the outer post surface and restricted penetration to the post interior. Without intending to be bound by theory, this observation is consistent with structures having an average mesh size of 2.09 nm, which are calculated based on the concentration of macromers in the pre-polymer solution using Flory-Rehner calculations and is predicted to effectively exclude the uptake of proteins with larger hydrodynamic radii (NA, M_{w} 60 kDa, RHYD 7 nm). As shown in FIG. 6, this measurement has been reaffirmed using additional fluorophore conjugates of globular proteins ranging in size that are predicted to either exhibit uptake or exclusion by posts upon rehydration. FIG. 6 illustrates mass inclusion/exclusion of rhodamine-conjugated proteins using hydrogel posts. For further characterization of mesh size, PEG-DA/AC-PEG-Biotin posts were dried and then rehydrated by the addition of PBS containing proteins of varying hydrodynamic radii conjugated to rhodamine (0-lactoglobulin A, M_{w} 18.4 kDa, hydrodynamic radius (RHYD) 2 nm; superoxide dismutase (SOD), M_{w} 32.5 kDa, RHYD 2.54 nm; ovalbumin, M_{w} 43-45 kDA, RHYD 2.92 nm). Fluorescent image exposures in column A (FIG. 6) were acquired 5 minutes after the addition of the protein-rhodamine conjugate. Images shown in column B (FIG. 6) were acquired following a subsequent wash with PBS (exposure times taken for each image are indicated). The absence of post fluorescence using ovalbumin and SOD is consistent with the absence of non-specific binding and their exclusion from the hydrogel matrix due to their respective molecular dimensions.

β-lactoglobulin A, however, shows uniform penetration, which is again evidenced by the calculated average mesh size for posts. The retention of fluorescence at the exterior of post surfaces following a brief wash (FIG. 6, column B, 40 ms exposure) is enhanced by the longer exposure time and was found to be absent subsequent to extensive washing with PBS.

FIG. 5B illustrates the absence of non-specific protein binding utilizing pA/G-rhodamine as a reporter, as well as NA-rhodamine blocked with saturating levels of biotin prior to its use in the assay (data not shown).

Without being bound by theory, FIG. 5C is a schematic illustrating fluorescence capture at outer post surfaces via biotin-NA binding interactions. Due to the post (or hydrogel feature) outer surface binding location, the presence of analyte can be more easily detected. FIG. 5D is an illustrative comparison of convective flow-mediated capture rate to a rate obtained for mass diffusion. Both plots are the average of 3 replicate assays and error bars have been removed so as to illustrate the differences in responses. FIG. 5D further illustrates the kinetics of convective flow-mediated NA binding over 4 hours, which exhibits a hyperbolic response highlighted by an initial rapid rate of analyte recognition that transitions to a greatly reduced rate at approximately 10 to 15 minutes following post rehydration.

As shown in Table 1, first derivative calculations of the slope for each time (t) value of the plot show the highest slope value at 1 minute (slope of 19.25), which diminishes over the time course according to a ln function (slope of 0.08 at 4 hours). In contrast, the kinetics of a mass diffusion control experiment performed in parallel is characterized as a second-order polynomial function with a slope of 0.18 at t=1 minute that decreases to 0.06 at t=4 hours. It is believed that the plot inset is representative of the time at which binding facilitated by convective flow transitions to binding by mass diffusion and that such a transition occurs within approximately 6 to 8 minutes following post rehydration.

TABLE 1

| Time (minute) | Convection | Mass Diffusion |
|---|---|---|
| 1 | 19.25 | 0.184 |
| 3 | 6.42 | 0.182 |
| 5 | 3.85 | 0.180 |
| 7 | 2.75 | 0.178 |
| 10 | 1.92 | 0.175 |
| 20 | 0.96 | 0.165 |
| 30 | 0.64 | 0.155 |
| 60 | 0.32 | 0.125 |
| 120 | 0.16 | 0.065 |
| 240 | 0.08 | −0.055 |

As described, Table 1 illustrates first derivatives (f′(x)) of convection and mass diffusion plots for absolute fluorescence units recorded as a function of time. Convection: (hyperbolic plot, described as y=19.247 ln(x)+14.126), R²=0.975). Mass Diffusion: (second order polynomical plot, defined as y=−0.0005x²+0.195+0.654, R²=0.930).

Without being bound by theory, in addition to facilitating a rapid acceleration of assay time, it is believed that convective flow, along with targeted improvements in experimental design parameters, are effectively harnessed for capturing considerably more analyte by mitigating the effect of the temporally diminished concentration gradients that reflect analyte depletion and drive diffusive mass transfer. In developing an appropriate model for comparing the kinetics of NA binding by convective flow versus mass diffusion, the mass diffusion assay was conducted by first rehydrating posts in PBS buffer, after which the appropriate volume of NA-rhodamine utilized for matching the concentration used in the convection assay was injected into the chamber. Gentle mixing of the chamber subsequent to the delivery of NA-rhodamine ensured a uniform concentration of NA-rhodamine.

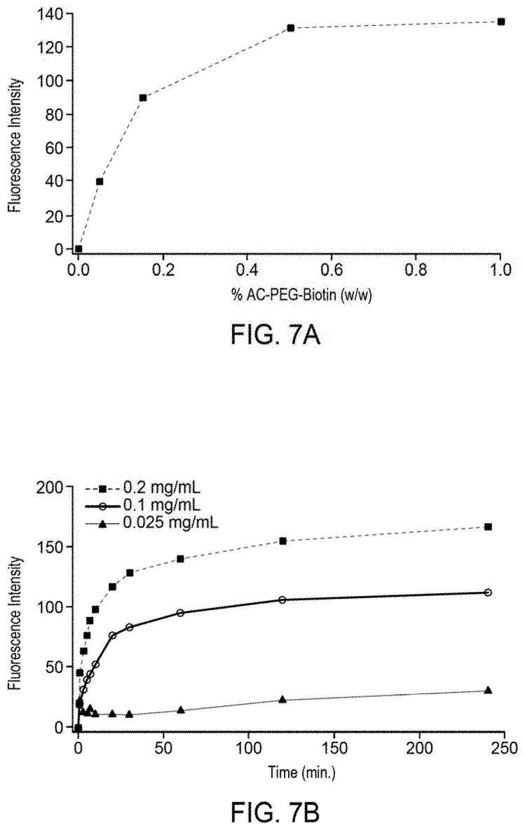

Parameters which influence convection-driven assay performance include variables that change in the matrix density of biotin and effect the relative level of NA capture are measured in a series of assays using hydrogels posts synthesized with 20% (w/w) PEGDA 700 and varying concentrations of AC-PEG-Biotin. FIG. 7A is a graph illustrating the impact of PEG-DA and AC-PEG-Biotin pre-polymer stoichiometry on NA-rhodamine capture and detection. The data represented in FIG. 7A was derived from a single assay for each concentration of AC-PEG-Biotin tested. As shown in FIG. 7A, the summary of these results shows that NA capture, as indicated by relative fluorescence intensity, rises as the concentration of biofunctional macromer is increased in pre-polymer solution and that a maximum is achieved at a concentration of 0.5% (w/w). Without intending to be bound by theory, this demonstrates the advantages of pre-determining a formulation for relative macromer concentration, which facilitates enhanced detection sensitivity by virtue of maximizing the availability of analyte recognition elements and/or minimizing diminished analyte recognition due to steric hindrance considerations.

FIG. 7B is a graph illustrating the detection rates for range of NA-rhodamine concentrations. The data represented in FIG. 7B is the average of 3 replicate assays for each concentration NA-rhodamine tested. As shown in FIG. 7B, subsequent detection sensitivity experiments utilizing hydrogel posts synthesized in accordance with this formulation exhibited fluorescence intensity profiles that reflect a roughly linear capture response with respect to a range of NA-rhodamine concentrations tested.

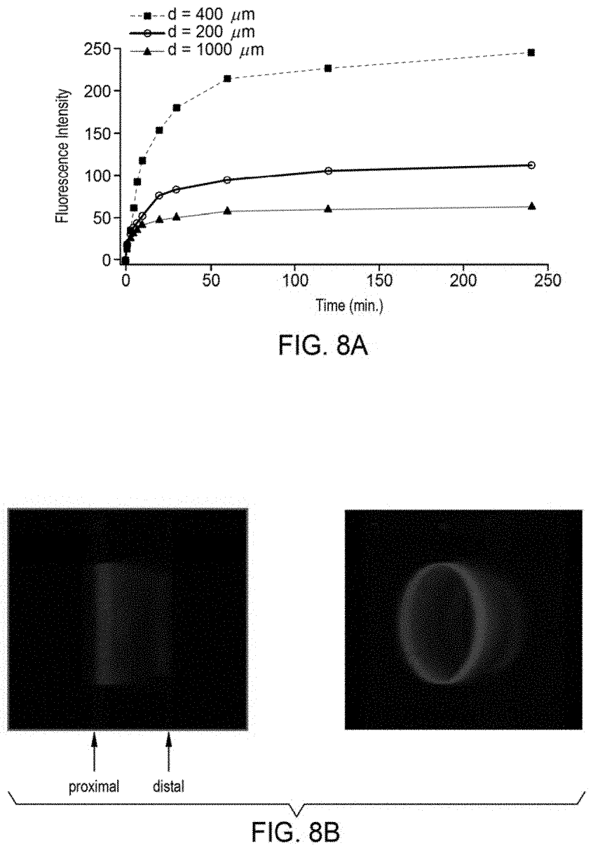

To identify additional assay parameters that can be tuned to improve detection sensitivity, hydrogel posts were synthesized with varying diameters, such as volume and functional surface area. FIG. 8A is a graph illustrating a plot of NA-rhodamine detection over time for hydrogel posts varying in diameters of 100 μm, 200 μm and 400 μm. The plot for each diameter is the average of 3 replicate assays. FIG. 8A shows that fluorescence intensity measurements for images captured in the x-y plane scale proportionately with changes in post diameter. Similar results were obtained using posts synthesized with a diameter of 200 μm in PDMS chambers of varying depths.

FIG. 8B is 3-D confocal scanning images of PEG-DA/AC-PEG-Biotin post incubated with NA-rhodamine. The posts are 200-μm diameter hydrogel posts using point-scanning confocal microscopy. In the left image, the post has been rotated 90 with respect to the z axis. In the right image, the post is rotated an additional 45 from the rotation position of the left image. Images were assembled from point scanning of 2.5 μm interval slices along the z axis using a 10× objective and a dwell time of 5.8 s. These results confirmed the reproducible synthesis of truncated structures of ˜140 μm in height that are convex at their distal termini and display a progressively diffuse decoration of fluorescence along the z-axis subsequent to incubation with NA-rhodamine

Without being bound by theory, the aforementioned results suggest a reduction in the biotin-functionalized matrix density that is attributed, to some extent, to diminished in-depth UV penetration beyond the glass slide support for anchored post synthesis. However, it is believed that attenuated hydrogel synthesis is a consequence of the interplay of several factors. Such factors can include UV power, UV exposure time, and the inhibitory effect of O₂ on macromer polymerization that is persistent despite efforts at N₂ purging. It is contemplated that various processing parameters and conditions may be modulated to fabricate hydrogel post structures with micro-architectural design features that enhance bioanalyte detection.

The versatility of convective flow-mediated hydrogel assays for enabling a range of biosensing applications were demonstrated using antibody grafted posts. In some embodiments, assays were conducted using posts synthesized from pre-polymer solution containing 20% (w/w) PEGDA 700 and 1% (w/w) AC-PEG-NHS. Following synthesis and washing to remove unreacted macromers, post fabrication entailed incubation with purified rabbit IgG to form stable amide bonds via the reaction of NHS groups and the primary amines of IgG, and then treatment with ethanolamine to block unreacted NHS groups. Data acquired for assays using IgG-functionalized posts and pA/G-rhodamine as a reporter shows (also shown using biotin-functionalized posts) intense fluorescence concentrated at the exterior surface of the post. In other embodiments, penetration to the post interior is more evident. Without being bound by theory, a higher level of porosity is believed advantageous when considering by the lower M_{w} of pA/G (i.e. 47.5 kDa) with respect to NA.

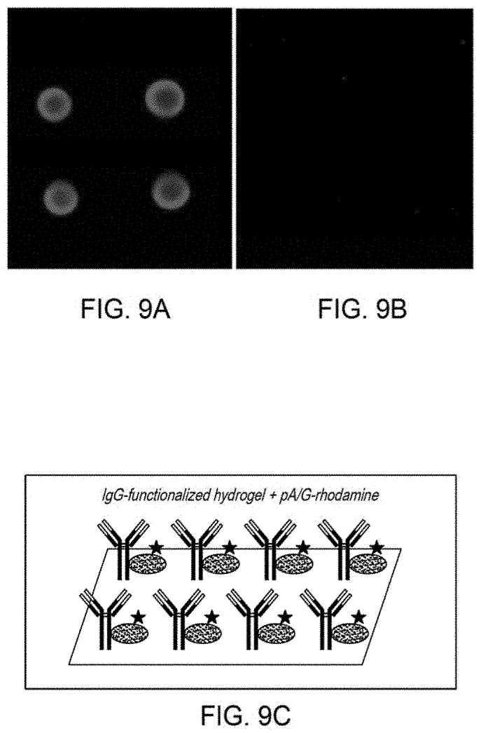

FIG. 9A is a fluorescent image of PEG-DA (20% (w/w))/AC-PEG-NHS (1% (w/w)) hydrogel posts functionalized with IgG and rehydrated in PBS containing 0.65 mg/mL pA/G-rhodamine (500 ms exposure acquired 5 minutes subsequent to initiation of reaction). FIG. 9B is an image of non-functionalized PEG-DA/AC-PEG-NHS posts rehydrated in PBS containing 0.65 mg/mL pA/G-rhodamine (500 ms exp.). As shown in FIG. 9B, the absence of non-specific surface interactions was demonstrated using non-functionalized posts and pA/G-rhodamine as a reporter. In some embodiments, the absence of non-specific surface interactions was demonstrated using IgG-functionalized posts using NA-rhodamine reporter. FIG. 9C is a schematic illustrating fluorescence capture at outer post surfaces via IgG-functionalized hydrogel posts and pA/G-rhodamine.

As described herein, a novel and economical approach for rapid analyte detection utilizes rehydration of microfabricated hydrogels and concomitant convective flow-mediated delivery of target analyte(s) to recognition elements as a platform for analyte detection. It is contemplated that assay features can improve the limits of detection sensitivity by utilizing micro-engineered increases in effective surface area capture via the development of hydrogel matrices with controlled pore dimensions for selective analyte uptake and binding, as well as utilizing nanocomposite capture matrices which include functionalized nanoparticles encapsulated within hydrogel scaffolds. Analyte detection platforms fabricated according to the embodiments described herein maintain and/or improve assay speed and sensitivity in detecting bioanalytes present in clinically relevant matrix test solutions (e.g. sandwich-type immunoassays).

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a surface-functionalized hydrogel feature” include aspects comprising one, two, or more surface-functionalized hydrogel feature, unless specified to the contrary or the context clearly indicates only one surface-functionalized hydrogel feature is included.

The term “coupled” is used herein to refer to elements that are either directly connected or connected through one or more intervening elements.

While the foregoing is directed to embodiments of the present disclosure, other and further embodiments of the disclosure may be devised without departing from the basic scope thereof, and the scope thereof is determined by the claims that follow.

What is claimed is:

1. An apparatus for analyte detection, comprising:
a substrate comprising a fluidic channel; and
a hydrogel structure disposed in the fluidic channel, the hydrogel structure comprising one or more surface-functionalized hydrogel features, wherein:
at least one of the one or more surface-functionalized hydrogel features comprises a chemically-bound probe, the chemically-bound probe configured to bind an analyte; and
the hydrogel structure is at least partially dehydrated.

2. The apparatus of claim 1, wherein a first surface-functionalized hydrogel feature of the one or more surface-functionalized hydrogel features comprising a first chemically-bound probe, the chemically-bound probe configured to bind a first analyte.

3. The apparatus of claim 2, wherein a second surface-functionalized hydrogel feature of the one or more surface-functionalized hydrogel features comprising a second chemically-bound probe, the second chemically-bound probe configured to bind a second analyte.

4. The apparatus of claim 3, wherein the second analyte is a non-specific analyte.

5. The apparatus of claim 1, wherein the chemically-bound probe is bound to the at least one of the one or more surface-functionalized hydrogel features by an acrylate group or a modified acrylate group.

6. The apparatus of claim 1, wherein the analyte is chemically bound to a fluorescent tag.

7. The apparatus of claim 1, wherein the analyte is chemically bound to a material detectable by Raman spectroscopy, surface enhanced Raman spectroscopy, or a combination thereof.

8. The apparatus of claim 1, wherein at least a portion of the apparatus is encased in a housing.

9. An apparatus for analyte detection, comprising:
a first component, comprising:
a substrate comprising a fluidic channel;
a hydrogel structure disposed in the fluidic channel, the hydrogel structure comprising one or more surface-functionalized hydrogel features, wherein:
at least one of the one or more surface-functionalized hydrogel features comprises a chemically-bound probe, the chemically-bound probe to bind an analyte; and
the hydrogel structure is at least partially dehydrated; and
a second component comprising a detector to detect the presence or absence of a material detectable by Raman spectroscopy.

10. The apparatus of claim 9, wherein:
a first surface-functionalized hydrogel feature of the one or more surface-functionalized hydrogel features comprising a first chemically-bound probe, the chemically-bound probe configured to bind a first analyte; and
a second surface-functionalized hydrogel feature of the one or more surface-functionalized hydrogel features comprising a second chemically-bound probe, the second chemically-bound probe configured to bind a second analyte.

11. The apparatus of claim 9, wherein the analyte comprises an analyte indicative of SARS-CoV-2, a spike protein of SARS-CoV-2, a nucleoprotein of SARS-CoV-2, or a combination thereof.

12. The apparatus of claim 9, wherein:
a first surface-functionalized hydrogel feature of the one or more surface-functionalized hydrogel features comprising a first chemically-bound probe, the chemically-bound probe configured to bind a first analyte; and
a second surface-functionalized hydrogel feature of the one or more surface-functionalized hydrogel features comprising a second chemically-bound probe, the second chemically-bound probe configured to bind a second analyte.

13. The apparatus of claim 12, wherein the second analyte is a non-specific analyte.

14. The apparatus of claim 9, wherein the analyte is chemically bound to the material having a Raman spectrum.

15. A method of detecting an analyte, comprising:
introducing a sample to an apparatus, the sample comprising an analyte, the apparatus comprising:
a substrate comprising a fluidic channel; and
a hydrogel structure disposed in the fluidic channel, the hydrogel structure comprising one or more surface-functionalized hydrogel features, wherein:
at least one of the one or more surface-functionalized hydrogel features comprises a chemically-bound probe, the chemically-bound probe to bind the analyte; and
the hydrogel structure is at least partially dehydrated;
exposing the one or more surface-functionalized hydrogel features to the sample; and
concentrating the analyte adjacent to or on the one or more surface-functionalized hydrogel features.

16. The method of claim 15, further comprising detecting the analyte by fluorescence spectroscopy, Raman spectroscopy, or a combination thereof.

17. The method of claim 15, further comprising reacting, under reaction conditions, the analyte and a fluorescent tag prior to introducing the sample to the apparatus.

18. The method of claim 15, further comprising reacting, under reaction conditions, the analyte with a material detectable by Raman spectroscopy prior to introducing the sample to the apparatus.

19. The method of claim 15, wherein the analyte is indicative of a virus.

20. The method of claim 15, further comprising detecting a non-specific analyte.
